(19) United States
(12) Patent Application Publication
(10) Pub. No.: US 2025/0195157 A1
(43) Pub. Date: Jun. 19, 2025

(54) PREDICTIVE CONTROL OF ROBOTIC MANIPULATOR FOR A CATHETER

(71) Applicant: Siemens Medical Solutions USA, Inc., Malvern, PA (US)

(72) Inventors: Young-Ho Kim, West Windsor, NJ (US); Florin-Cristian Ghesu, Baiersdorf (DE)

(21) Appl. No.: 18/538,230

(22) Filed: Dec. 13, 2023

(51) Int. Cl. A61B 34/30; A61B 17/00; A61B 34/00; B25J 9/16

(52) U.S. Cl. CPC A61B 34/30; A61B 17/00234; A61B 34/71; B25J 9/163; A61B 2017/00318; A61B 2034/301; A61B 2034/302

(57) ABSTRACT

For predictive control of tendon-driven continuum mechanisms (TDCMs), a machine-learned model predicts future control of the motor or robot based on user commands to move the catheter. For example, a robotically operated catheter includes a TDCM. The machine-learned model, such as a recurrent neural network or another artificial intelligence, predicts future control. This prediction may account for the unknown environment using input of past states of the motor and/or position of the tip of the catheter or other steered device.

### BACKGROUND

The present embodiments relate to predictive control of a robotic manipulator for a catheter or another device with a tendon-driven continuum mechanism (TDCM) (e.g., endoscope). TDCMs are used in medical devices that are inserted through a narrow and tortuous path within a patient. The TDCM may be a long and flexible hollow pipe acting as a sheath and a wire inserted into the pipe acting as the tendon. When the wire is pulled at one end, the wire slides within the sheath so that the pulling force is transmitted to the other end of the sheath. The TDCM has non-linear behavior caused by elasticity, slack, backlash hysteresis, and non-linear friction between the sheath and the wire. These phenomena degrade the control accuracy. Pre-defined control cannot handle the dynamic environmental changes in the patient over time.

Various attempts have been made for modeling this non-linear behavior. Some models account for the bending shape in 3D space, which involves mapping the tendon lengths to the corresponding tip position and orientation. A simplified approach uses a constant curvature assumption in closed-form forward kinematics. Due to the high nonlinearities and the indefinable uncertainties caused by the unknown arrangement of the TDCM in a patient, an accurate analytical model is hard to derive. These errors may be compensated by sensor feedback (electromagnetic sensor, optical sensor, load cells), but such sensors have practical limitations (sterilization, cost, and size).

To reflect dynamic characteristics, there exist several works related to friction/hysteresis compensation in tendon-driven manipulators. Various mathematical models model hysteresis but involve many hyperparameters and are associated with complicated identification procedures. These existing methods are not adaptive due to environmental changes associated with TDCM in medical devices. Model predictive control (MPC) is an advanced method of process control while satisfying a set of constraints. MPC has been used in many autonomous systems (e.g., self-driving car). If the problem complexity is increased (e.g., non-linearity), MPC needs a longer time to handle. Moreover, MPC requires a proper model of the system to be stable and needs a periodic observation to update in order not to diverge. The TDCM manipulation does not have a direct observation for the desired pose controls in real time and involves process noise and delay, making MPC difficult to use with TDCM.

### SUMMARY

By way of introduction, the preferred embodiments described below include methods, systems, non-transitory computer readable storage media with instructions, and robots for predictive control of TDCM, such as robotically operated catheters. A machine-learned model, such as a recurrent neural network or another artificial intelligence, predicts future control of the motor or robot based on user commands to move the catheter. This prediction may account for the unknown environment using input of past states of the motor and/or position of the tip of the catheter or other steered device.

In a first aspect, a method is provided for robotic control of a TDCM. A user command to operate the TDCM is received. An artificial intelligence predicts a motor control. The artificial intelligence predicts in response to input of the user command. A motor is operated with the motor control. The operating of the motor operates the TDCM.

In a second aspect, a control system is provided for a steerable catheter. A robotic manipulator is provided for operation of the steerable catheter. The robotic manipulator includes an actuator configured to steer the steerable catheter. A control processor is configured to control the actuator. The control uses application of a machine-learned model configured to predict a position and/or velocity of the actuator to implement a user command given an environment of the steerable catheter.

In a third aspect, a method is provided for predictive control of a robotic manipulator for a catheter. A machine-learned model predicts a sequence of future states of operation of a motor of the robotic manipulator based on a sequence of past states of the motor operation. The motor of the robotic manipulator is controlled based on at least one of the predicted future states.

Additional aspects and features are summarized below as the illustrative embodiments. The present invention is defined by the following claims, and nothing in this section or the illustrative embodiments should be taken as a limitation on those claims. Features of one aspect or type of claim (e.g., method or system) may be used in other aspects or types of claims. Further aspects and advantages are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a flow chart diagram of one embodiment of a method for robotically controlling a TDCM;

FIG. 2 illustrates training (off-line) and application (on-line) phases for a machine learning model to predict controls for a robot operating a TDCM;

FIG. 3 illustrates example controls of a catheter using TDCM; and

FIG. 4 is a block diagram of one embodiment of a control system for a steerable catheter.

### DETAILED DESCRIPTION OF THE DRAWINGS AND PRESENTLY PREFERRED EMBODIMENTS

TDCM-based catheter or medical device (e.g., endoscope) structures are in a narrow and tortuous environment during a procedure. During the procedure, the flexible shaft shape is arbitrarily changed, leading to a dynamic change of each tendon tension condition. For better control, the goal is to identify changes of tendon tension in unknown shapes of the proximal shaft and adaptively identify parameters for a compensated motion that minimize errors. The technical challenges are how to deal with multi-dimensional sequences and predict dynamical change of states over time.

Predictive control of TDCM is provided. An artificial intelligence as a real-time model provides a long-term horizon state estimation for predictive control. The predictive control identifies change of control parameters due to environmental interactions. State information from robotic manipulators (e.g., motor encoder, time-series motor current, model-based kinematics, hysteresis, etc.) is used to train the artificial intelligence (e.g., learning-based model). A long-term (e.g., multiple steps or over multiple seconds) horizon state estimation makes an automated safe navigation system for TDCM. The estimation over time may also provide a quantification of uncertainty in motions. The use of the artificial intelligence may result in less restrictive or precise calibration being needed, facilitating calibration in a short time.

In one approach, the artificial intelligence is used to fine-tune motor control. The artificial intelligence identifies and predicts long-term predictive states based on time-series historical motor states and future expected motion sequence during operation, which can be integrated with the existing kinematics/hysteresis model. The kinematics/hysteresis model controls the motor in response to user input, with the artificial intelligence offsetting the control to account for the environment (e.g., curvature) of the TDCM. This is useful for a safe motion control for automated navigation.

The artificial intelligence-based motor control may be used for various medical devices with unknown curvature and/or environment within a patient, such as steerable catheters and endoscopes. Any device using tendon, cable, or geared steering may be used. The steerable catheter example will be used herein. The artificial intelligence-based predictive motor control may be used with an intracardiac echocardiogram catheter (ICE). Where more than one TDCM is provided in a given medical device, the same or different artificial intelligence is used in the control of each TDCM. Due to the predictions, the calibration procedures of the catheter may be simplified as the artificial intelligence provides accurate tip control with less precise calibration, facilitating autonomous navigation of catheter.

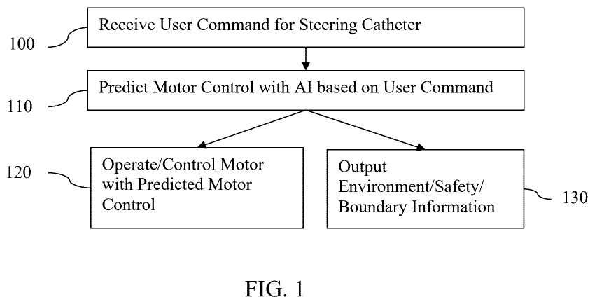

FIG. 1 is a flow chart diagram of one embodiment of a method for robotic control of a TDCM, such as control of a robotic manipulator for a catheter. The control uses an artificial intelligence or other machine-learned model to predict future motor controls. To account for the unknown environment (e.g., amount of friction due to curvature), the prediction provides motor control to more likely result in the TDCM steering more precisely to the user command. For example, the user command is to rotate the tip by 3 degrees. Due to friction, the motor may need to move 5 degrees. The artificial intelligence predicts the extra (2 degrees), total (5 degrees), or another adjustment to provide the desired operation.

The method is implemented by the control system and/or robotic system of FIG. 4 or another system. A processor receives the user command, predicts motor control with the artificial intelligence, operates the motor using the prediction, and/or outputs information based on the prediction. The robotic catheter system moves the catheter based on the motor control. An imaging system may use an array or other sensor on the catheter to generate and display an image of the patient.

Additional, different, or fewer acts may be provided. For example, act 130 is not provided. As another example, acts for the catheterization workflow are provided. In yet another example, acts for pre-operative planning, calibration, and/or imaging are provided. As another example, acts for ablation, stent placement, or another catheter function are provided.

The acts are performed in the order shown (top to bottom or numerical) or a different order. For example, act 130 occurs during (e.g., simultaneously or in real time) or before act 120.

In act 100, the control processor receives a user command to operate the TDCM. The user inputs with a user input device, such as a knob, joystick, mouse, track ball, touch pad, keyboard, buttons, or other input. The input may be in real-time, such as the user inputting to immediately control the catheter. The input may instead be off-line, such as the user pre-planning catheter operation, and the control processor then implementing the plan. In other approaches, the command for movement is from image processing, such as a machine-learned model determining movement to automatically perform a medical procedure.

The user command is for steering the catheter. The command is to rotate the catheter along one or more axes. The command may instead, or additionally, be to translate the catheter along one or more axes. For example, the user desires to rotate the body of the catheter about the longitudinal axis, translate along the longitudinal axis, and/or bend the catheter. The user inputs a command to rotate, bend, and/or translate and by how much and which direction. Any command to steer or move the catheter is input.

The TDCM-based device (catheter) is motorized. The tendon or other force applying mechanism of the TDCM is controlled by an actuator (motor). The user command indicates movement of the catheter, and a kinematics model translates the catheter movement to motor movement (which motor to operate and by how much). The kinematics and/or hysteresis model may be used to translate the user command in catheter space to motion by the motor or motors.

Due to the environment of the catheter, the control by the kinematics and/or hysteresis model may be inaccurate. The catheter is subjected to an unknown amount of bending or curvature and/or external pressure. This environment may vary patient-to-patient, procedure-to-procedure, and/or during a procedure for a given patient. As a result, a greater tension and/or friction is applied to the tendon or TDCM to overcome environmental effects. Different amounts of motor movement may not linearly map to different amounts of actual steering or catheter movement.

In act 110, the artificial intelligence predicts a motor control. The artificial intelligence predicts in response to input. Various inputs may be provided. In one approach, the input is the user command, images, motor parameters (e.g., current, position, velocity, and/or torque), and/or catheter position (e.g., tip position detected from imaging and/or electromagnetic position sensing). For example, the inputs are motor parameters and the user command.

The input may be past and/or current values. For example, ten or more past and the current values are input. As another example, values from the past X seconds (e.g., X=1-60) and from the current time are input.

The prediction is for motor control for a next change or a sequence of next changes. For example, one or more future motor controls are predicted. The number of future motor controls may be for any amount of time, such as over 5, 10, or 20 seconds or number of time increments, such as 1-10.

The artificial intelligence is a machine-learned model. Any machine-learned model may be used, such as a neural network. Neural networks include fully connected networks, convolutional networks, or others. In one approach, a recurrent neural network, such as a neural network with long-short-term memory, a transformer, or neural ordinary differential equation network (N-ODE), is used. Any network receiving a temporal sequence as input to output an action, decision, and/or sequence of actions or decisions may be used.

For a recurrent neural network, the machine-learned model was previously learned using reinforcement learning. A reward system is used to learn how to predict motor control through a series of actions. The training learns a policy indicating how to control the motor through a sequence of optimizations. Reinforcement learning may be used to learn the optimal or a variety of actions for motor control. Machine learning techniques are used to automatically identify the best or other options for how to control the motor for TDCM among the available alternatives. The reinforcement learning learns a “policy” (e.g., a guideline of how to optimize the motor control). Because different actions may be used (e.g., number of steps, amount of motion, and/or other motor control), the motor control may be encoded as a Markov decision process. An optimal policy may be computed from the Markov decision process using, for example, dynamic programming or more advanced reinforcement learning techniques such as Q-Learning. During the learning procedure, an agent repeatedly tries different actions from the set of available actions to gain experience, which is used to learn an optimal policy. A policy determines for any possible state during the decision process the best action to perform to maximize future rewards. The rewards are set up in a way such that positive rewards are given for actions that lead to desired motor control (e.g., accurate motion and/or low execution time), while negative rewards are given for experiments which provide little or no value to the decision-making process. Only positive or only negative rewards may be used. Experience from past decision-making processes may be used to define the rewards and the states. For application, this learnt policy is applied without measuring reward.

For training, the training data includes many samples. The samples are a dataset with inputs and corresponding known desired motor control as the ground truth. The deep learning learns features to be extracted from the dataset. These learned features are to be used by the learned policy. The features that may be used to distinguish between actions best or sufficiently are learned from the training data. For example, deep learning (e.g., deep structured learning, hierarchical learning, or deep machine learning) models high-level abstractions in data by using multiple processing layers with structures composed of multiple non-linear transformations, where the input data features are not engineered explicitly. A deep neural network processes the input via multiple layers of feature extraction to produce features used to predict motor control. Other deep learned, sparse auto-encoding models may be trained and applied.

For training, action embedding in combination with policy gradient is used to exploit the intrinsically sparse structure of the relevant action space. In another embodiment, natural evolution strategies (NES) perform the policy exploration in parameter space as part of the learning. NES is particularly suited given the compact parametrization of fully convolutional networks. Other approaches for deep reinforcement learning may be used, such as Q-learning.

The reward system may be defined using a difference from the ground truth. The overlap of the output motor control with the ground truth is determined in training. If overlapping, a reward is given. Otherwise, no reward is given. The overlap may be measured in linear or non-linear relationship of direction, magnitude, and/or velocity of motor operation. Binary or continuous reward parameters may be used. Other reward functions may be used, such as chi-square, Dice, or other measures of amount of difference.

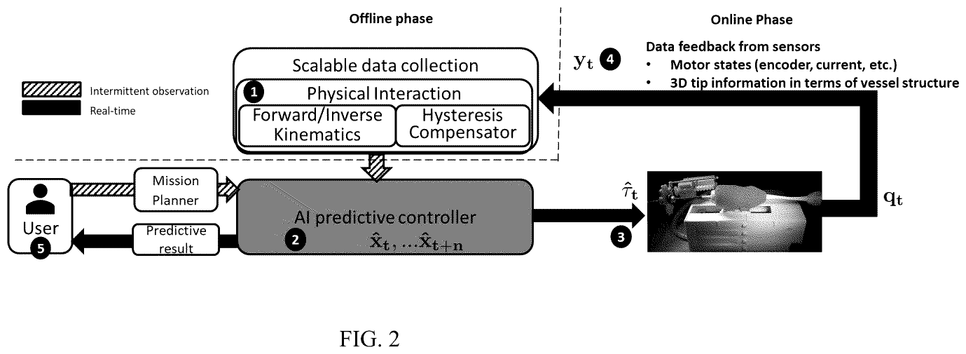

FIG. 2 illustrates one example approach for scalable learning. A scalable learning pipeline is provided for artificial intelligence-based predictive control toward autonomous TDCM-based catheter manipulation. The pipeline shown in FIG. 2 has two phases: an offline phase, and an online phase. The offline phase corresponds to training. The online phase corresponds to application of the trained policy. In the offline phase, scalable data collection is performed at (1) in two ways; one from physics-based simulator and another from a test jig. In the online phase, the user control center (5) provides user commands to a predictive control (2) including a long-term state estimator for control of the robotic manipulator (3) based on data feedback from sensors (4).

For training, the scalable data collection provides the training samples, including ground truth used for rewards. Physical interaction data is important to get a better quality of predictive control. Data corresponding to intended and actual movement of the catheter is acquired.

The training data may be obtained by monitoring procedures performed on patients. The motor states are obtained with sensors, the catheter states obtained with sensors or imaging, and the user command obtained from input. In another approach, scalable data collection uses a physics-based simulator and/or a test jig. Simulation and/or test jigs form scalable environments for device control. The physics-based simulator may be integrated with pre-operative images, which can provide varied environmental structures with various motion controls. The physics-based simulation models the procedure in the environment given by the imaging. For the test jig, an adjustable tube structure models the environment. The test jig can be manipulated to any environmental contact. The change for environmental constraints (e.g., obstacles, curvy corridors, etc.) is the proximal shaft shape. To get the scalable data, the outer shape of the jig is manipulated while collecting data over time. Physical properties of the internal interaction in the TDCM are finite and collected using a simple setting. The catheter is robotically controlled within the test jig. Various states of device motions are created in the scalable environment setting (test jig and/or simulation).

The training data is collected. In one approach, the input data is a number N of tendon tensions, tendon traveled distance, associated sequential motions, and 3D position of the tip with electro-magnetic sensors. Other input data may be gathered. The resulting motion of the tip in response to input commands or deviation from desired motion is collected, forming the ground truth.

The training provides a deep reinforcement machine-learned model as the artificial intelligence. This model is trained to predict motor controls. The motor and/or catheter information (e.g., past and current states) are input, and the model outputs the motor control(s).

In act 110, the control processor applies the artificial intelligence as trained. The learned model defines an action space. The learnt policy of the model provides an action or actions for each iteration of motion control. The action changes some aspect of the motor. The machine-learned policy controls the sequence of predictions for actions (motor control). The policy, as the artificial intelligence, provides decision making for predicting motor controls given past and/or current states. The refinement as motor control is described by state change. The actions are for change of state.

The prediction is a motor control corresponding to a change in the motor state. The motor control accounts for the friction, elasticity, slack, and/or other environmental conditions caused by a current curvature and/or placement of the TDCM in the patient.

For making the prediction, input values are applied to the artificial intelligence. For example, covariates and/or states for the motor and/or catheter are input. The current draw on the steering motor, distance of insertion within the patient provided by an encoder of the motor, motor position, motor velocity, motor torque, and/or other motor characteristics are input with the user command. Catheter states include the tip location and/or velocity. The tip may be an end region of the catheter, such as where the TDCM in anchored on the catheter. The anchor position may be used for the state in other approaches. An combination of inputs may be used, such as motor operation parameters and user command without catheter position or velocity. A temporal sequence of such values (e.g., motor position, motor velocity, motor current, and/or distance from an encoder) are input. Any number of past states or covariates may be used, such as tens or hundreds.

In response to input at a given time (e.g., input of current and past states), the artificial intelligence (e.g., machine-learned model) predicts one or more (e.g., a sequence) of future states of operation of the motor of the robotic manipulator. For example, the recurrent neural network includes a memory that stores the most recent past states to output a sequence of multiple motor controls as actions for motor motions. Where cost or reward are used in the training, the policy generating the actions minimizes the cost or maximizes the reward in the predicted motor controls to implement the user command. The prediction may account for any constraints, such as avoiding collusion with or pressure on anatomy. The artificial intelligence predicts future states as a sequence of actions in response to past states and a current state.

In one approach for artificial intelligence predictive control, x(t) is defined as a three-dimensional (3D) tip position at time t. q(t) is the motor state (e.g., position and velocity) to apply at time t. Given the current position A and desired location B to go of the tip, the prediction is to create the best sequence of motor motion minimizing the object cost (i.e., execution time and/or accurate motions) under constraints (i.e., no collision with the environment). This problem is in duality: one for forward state estimation, which estimate the forward kinematics including hysteresis; another is for inverse kinematics, which estimate the next motion to apply. A long-term state is estimated based on traces (e.g., the history of sequential states). The recurrent neural network (e.g., RNN, LSTM, Transformer, N-ODE, etc.) models the long-term state estimator. The future states are estimated from the past state, past covariates, and/or known future covariates. The past state is 3D tip position x(1, . . . t), and the future state is x(t+1, t+2, . . . t+k) where k is the number of the future time step. Past covariates are multiple variates including motor current, motor state (position, velocity) while future covariate is motor state only. After training, the future motions according to each future state minimizing the cost function are output. The created motion is applied to the robotic manipulator (3) based on data from sensors including motor states and 3D tip information as feedback (4). Other inputs may be used for training and/or application. For example, just the motor state (encoder, position, and/or velocity) is used without the 3D tip position of the catheter.

The prediction is for a fine-tuning of the motor operation. For example, an additional magnitude and/or direction is provided. This addition is provided for a next control to the motor with any control for other sources, such as the kinematics and/or hysteresis model. The kinematics and/or hysteresis model outputs control of the motor based on the user command. The prediction from the artificial intelligence is for additional control to deal with the environment to provide the desired catheter motion. In other approaches, the prediction is of motor control in the form of environmental factors, such as a level of friction, from which the position and/or velocity of the motor is derived. In yet another approach, the prediction subsumes the control from the kinematics and/or hysteresis model. The artificial intelligence predicts the motor state without being a fine tuning from another model. The output may be in catheter space (e.g., catheter motion) or in robotic space (e.g., motor motion).

In act 120, the motor operates with the motor control. For moving, the processor controls the motors of the robotic catheter system. The robotic catheter system, by actuation of the motors, manipulates the catheter. The manipulation may include steering (e.g., bending in one or more planes), global rotation (e.g., rotation of the handle and corresponding catheter), global translation (e.g., pulling or pushing the catheter further out or further into the patient), imaging, ablation, puncture, inflation, stitching, clamping, cutting, pharmaceutical deposit, stent expansion, balloon inflation, stent release, and/or another operation. The robot, under control of the processor, operates the catheter. Similarly, the user interface (e.g., display and input devices) of the robotic catheter system provides manual control of the robotic catheter system. The processor may receive the inputs and/or generate the outputs for the user interface and provide controls to the motors. The processor may generate controls for the motors based on programming for automated or semi-automated operation.

The motor is operated based on the predicted motor control for the next time step and/or over a sequence of time steps. The TDCM is operated by the motor using, at least in part, the motor control output by the artificial intelligence. One or more future states as predicted are used to control the motor. The motor position change and/or velocity is used to control the steering through the TDCM. Since the motor control accounts for the environment through the artificial intelligence prediction, the motor control more likely results in the desired (user commanded) motion of the catheter.

In one implementation, the motor is operated with the user command. The predicted motor control from the artificial intelligence is added to the operation based on the user command. This is a fine tuning of the motor operation using the prediction. For example, a kinematics and/or hysteresis compensator or model uses the user command and calibration to output the motor settings based on the user command. The kinematics and/or hysteresis model controls the motor. The one or more of the predicted future states are used to fine-tune the control by the kinematics and/or hysteresis model. The fine-tuning accounts for friction of a tendon of the catheter due to curvature of the catheter in a patient and/or another environmental factor. The motor settings from the kinematics/hysteresis model are adapted or altered to fine tune for the environment. The artificial intelligence outputs the change to the settings and/or replacement settings to fine tune. In another approach, the motor control from the kinematics and/or hysteresis model are input to the artificial intelligence with other inputs to predict the motor control accounting for the environment. In yet another approach, the output of the artificial intelligence is input to the kinematics and/or hysteresis model, which determines the motor control based, in part, on the output of the artificial intelligence.

The motor control is sent to the motor. Using position, velocity, torque, or another motor control process, the motor moves the tendon. The processor controls the robotic catheter system to move the catheter. For example, the processor causes the robotic catheter system to translate, rotate, and/or bend the catheter while part of the catheter is in the patient.

In one implementation, the motor control for the next time step is used to control the motor. Other future time step predictions may not be used for motor control or are used where the implementation of the change in positions commanded by the user is divided into multiple time steps. The artificial intelligence may update the motor controls less frequently than the time steps used to control the motor. The movement desired by the user is performed in two or more stages, so multiple predictions corresponding to the different stages are used to control the motor. The inputs to the artificial intelligence may be updated each time step and new outputs generated in real-time.

In act 130, additional information is derived from the prediction or predictions by the artificial intelligence and output as text, highlighting, or a warning. The additional information may be environmental information, safety information, and/or boundary information. By predicting future motor controls, the prediction over future times may indicate environmental, safety, and/or boundary information. Other outputs of other types of information may be generated based on the output of the artificial intelligence.

In one implementation, environmental information for the TDCM is output. The artificial intelligence outputs one or more (e.g., a sequence) future motor controls. These controls have magnitude for one or more motor operations, such as position and velocity. The artificial intelligence may also output a probability for each of the future motor controls (e.g., uncertainties in the position and velocity). The probability may be an indication of uncertainty. The magnitudes and/or uncertainty may indicate characteristics of the environment. For example, higher magnitudes may indicate greater curvature, a more tortuous path, and/or tighter bends. The magnitude may be compared to expected values due to pre-operative planning or imaging or default values. A mismatch may indicate that the environment is unexpected. High uncertainty may indicate unexpected environment. Combinations of magnitude and probability may be used. The output is compared to default thresholds or thresholds based on pre-operative planning. The output may be compared value-by-value, an average across a sequence, a maximum across the sequence, or a minimum across the sequence. A change or trend in the outputs may be used to reflect the environment. The output of the artificial intelligence is used as an interpreter to understand how the environmental structures are changed due to manipulation, which can quantify the uncertainty on the current controls. Environmental changes are reflected by the artificial intelligence as an encapsulated model, which represent environmental status. A notice of such environmental information is output to the surgeon.

In another implementation, safety information for the TDCM is output. The magnitudes and/or probabilities output by the artificial intelligence may be used to identify safety boundaries. The multi-variate time-series artificial intelligence model handles motor controls with a desired trajectory in unknown environmental changes. By monitoring output of the model, the motor controls can be safely applied into the safety boundary. For example, high uncertainty may generate a warning as the environment is unexpected. The warning indicates that the surgeon should be cautious to avoid patient harm. As another example, magnitudes over a threshold may indicate future contact with anatomy, so a warning is generated. A notice of such safety concern is output to the surgeon.

In yet another implementation, future boundary contact by the TDCM is output. The magnitude and/or probability output by the artificial intelligence may be used to identify contact with an anatomical boundary. The time-series motor currents reflect environmental changes, so the trend or change in motor controls over time show key points to detect a boundary. A sudden rise (e.g., threshold difference) in magnitude and/or uncertainty may indicate expected future contact with a boundary. A notice of such future boundary contact is output to the surgeon.

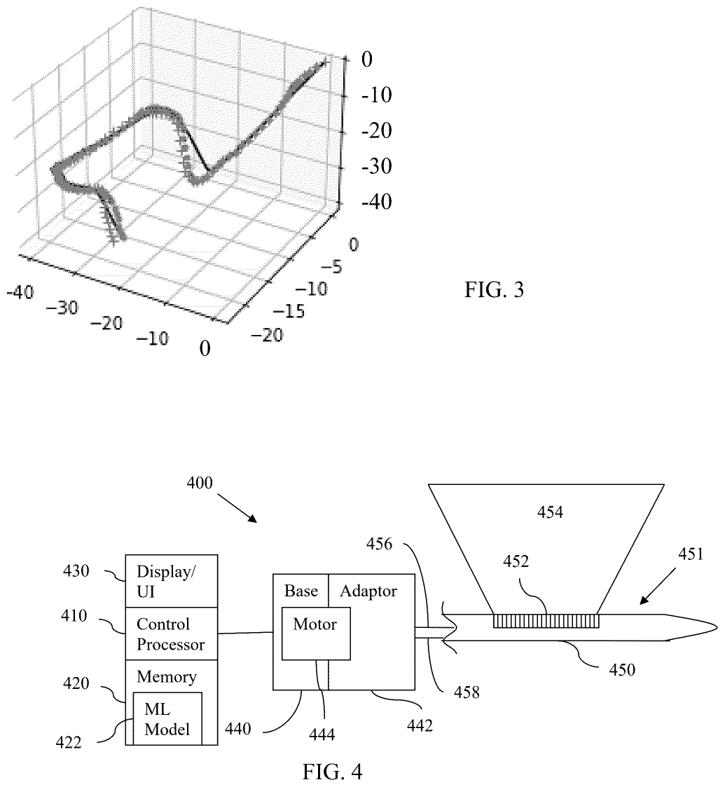

FIG. 3 illustrates an example of predictive motor control. The artificial intelligence estimates future states 18 seconds out given the past 24 states and the current state as input while pushing a catheter with 2 Nm of torque in a vessel. The solid line indicates the actual vessel centerline. The line of dots shows the ground truth. The plus is the estimated motor control value as a combination of a kinematics and hysteresis model with fine tuning by the artificial intelligence. Thus, the desired catheter tip position state is output based on applied compensated motor controls. In testing with 223 real testing data sets, 0.9 mm+0.22 mm for RMSE is achieved. The fine tuning by the artificial intelligence adds precision or accuracy in controlling the catheter.

The output of the artificial intelligence is used to control the motor of the robotic manipulator. The output motor control is converted into motor space (e.g., by kinematics) or is already in motor space. The output may also be displayed. Alternatively, or additionally, warnings or notifications derived from the output are displayed, such as the environmental, safety, and/or boundary information. Images, where the catheter is capable of imaging, may also be displayed. The display is on a screen or display device, such as a computer connected to or part of the robotic manipulator. For non-imaging catheters, the catheter is used to apply treatment (e.g., ablation, stenting, suturing, cutting, pharmaceutical application, or another) or assistance (e.g., clamping, moving tissue, or another).

FIG. 4 is a block diagram of one embodiment of a catheter control system. A control system using a robotic catheter system 400 is provided for a steerable catheter 450. The control system uses a machine-learned model 422 (artificial intelligence) to predict motor controls. Since the machine-learned model 422 is used, more accurate motor controls to provide the desired catheter movement are provided. The machine-learned model 422 was trained to operate with the catheter in different environments so accounts for the generally unknown environment of the catheter in the patient.

The control system includes the robotic catheter system 400 formed by the base 440 and adaptor 442. The control processor 410, memory 420, and/or display 430 are part of a separate computer, workstation, or server and/or are part of the robotic catheter system 400. Additional, different, or fewer components may be provided. For example, the base 440 and adaptor 442 are combined to be one housing for motors and clamps to manipulate the catheter 450.

The robotic catheter system 400 is configured for robotically controlling the catheter 450. In an example used herein, the robotic catheter system 400 is configured to control an ICE catheter 450. Other types of steerable catheters may be used, such as controlling an ablation catheter or another imaging catheter (e.g., endoscope). Other catheter-like devices (e.g., fiber optic endoscope or needle) may be controlled. These different catheters may have the same or different arrangements of control knobs, steering, degrees of freedom, and/or operation, so the robotic catheter systems 400 is configured to operate or control the arrangements of the catheter 450 being used. Different catheter systems may be configured to operate with different types of catheters and not other types.

Any type or design of robotic catheter system 400 may be used. In the example herein, the robotic catheter system 400 includes the base 440 and an adaptor 442. The base 440 includes the motors or actuators 444 for steering and/or operating the catheter 450, and the adaptor 442 is configured to mate with, hold, and/or manipulate the catheter 450. For example, the adaptor 442 allows the robotic catheter system 400 to operate the steerable ICE catheter 450 designed for manual control by a physician. U.S. Pat. No. 11,590,319, the disclosure of which is incorporated herein by reference, discloses various embodiments of such a robotic catheter system. Other designs may be used, such as robotic catheter systems designed to robotically control a catheter designed specifically for or to mate with and be controlled by the robot (e.g., no adaptor 442).

The base 440 includes the actuators or motors 444 to manipulate the steerable catheter 450 in any number of degrees of freedom, such as 4 or more degrees of freedom (e.g., global translation, global rotation, and bending or steering the catheter 450 in two or more directions or planes). Gearing, motors 444, grippers (clamps), connectors, and/or other robotic components are included the base 440 for generating and transmitting force to operate the catheter 450. The robotic catheter system 400 applies push and/or pull forces to steering wires 456, 458 (e.g., three or four steering wires or TDCM per catheter 450) to operate the catheter 450. The tips are steered for catheter operation. Electrical signals, translation, and/or rotation force may be generated in the base 440. Global translation and/or rotation forces may similarly be applied, such as through the handles of the catheter 450.

The adaptor 442 connects or plugs to the base 440 and manipulates the catheter 450. In one embodiment, the adaptor 442 is a clamshell or other arrangement of housing, gearing, connectors, clamps, and/or other robotic components to transfer force from the base 440 to the catheter 450 to steer and/or otherwise operate the catheter 450. In one approach, the adaptor 442 fits around the handle and/or body of the catheter 450 to allow for manipulating the actuators (e.g., knobs) of the catheter 450, for instance AP/LR knobs of an ICE catheter and/or the handle of the catheter 450 for global translation and/or rotation (e.g., push-pull knob of an ablation catheter, etc.). Other adaptors 442 that support other degrees of freedom (e.g., 2, 3, 4, or more) may be used, such as for control of needle or ablation catheters.

Where the steerable catheter 450 is an ICE catheter, the robotic catheter system 400 may be configured to control the ICE catheter, including imaging. The array 452 of elements is controlled through beamforming to generate ultrasound images. The field of view 454, type of imaging, and/or other imaging control may be provided. Where the steerable catheter 450 is an ablation or other treatment catheter, the robotic catheter system 400 may be configured to control the ablation or treatment catheter. For example, the contact sensing and/or electrode or another ablation applicator is controlled to ablate or treat. The ablation control may be performed by the robotic catheter system 400 and/or the processor 410.

The steering wires 456, 458 are TDCM or tendons that extend from the adaptor 422, a handle of the catheter 450, and/or motors (actuators) 444. The steering wires 456, 458 terminate at a tip 451 of the catheter. Different steering wires 456, 458 may terminate at the same distance or different locations (e.g., different distances along the longitudinal axis). The tip 451 may include a region with the array 452 or may be distal to the array 452. For non-imaging catheters, the tip may include or not include a working portion (e.g., stent region) of the catheter.

The memory 420 is a non-transitory memory, such as a removable storage medium, a random-access memory, a read only memory, a memory of a field programmable gate array, a cache, and/or another memory. The memory 420 is configured by a processor, such as the processor 410, to store the machine-learned model 422, kinematics and/or hysteresis model, motor control, values of motor parameters (e.g., position and velocity), catheter tip position, instructions executable by the processor 410, instructions executable by the robotic catheter system 400, interface software, and/or other information.

The display/user interface 430 is a display screen, such as a liquid crystal display or monitor, and a user input, such as a keyboard, knobs, sliders, touchpad, mouse, and/or trackpad. The display/user interface 430 may include icons, graphics, or other imaging, such as from an operating system or application, for user interaction (selection, activation, and/or output).

The processor 410 is a general processor, application specific integrated circuit, integrated circuit, digital signal processor, field programmable gate array, artificial intelligence processor, tensor processor, and/or another controller for interfacing with or controlling the robotic catheter system 400, the user interface/display 430, and/or catheter 450. The processor 410 is configured by design, hardware, firmware, and/or software to interface between the user and robotic catheter system 400 and/or to control the robotic catheter system 400. Multiple processors may be used for sequential and/or parallel processing as the processor 410. The instructions from the memory 420, when executed by the processor 410, cause the processor 410 to operate the robotic catheter system 400 and/or the steerable catheter 450 and to interface between various components and/or with the user or operator.

The processor 410 is in a same room as the robotic catheter system 400. For example, a computer or workstation is connected with wires or wirelessly for interacting with the robotic catheter system 400. In one implementation, the processor 410 is a processor of the robotic catheter system 400. Alternatively, the processor 410 is in a different room than the robotic catheter system 400, such as in a room of the same building, different building, different facility, or different region. Computer network communications are used between the processor 410 and the robotic catheter system 400. The control through the processor 410 for the robotic catheter system 400 allows the processor 410 to be fully remote from the procedure, patient, and/or robotic catheter system 400 as the processor 410 provides for the catheter 450 to be manipulated robotically.

The processor 410 is configured to cause the robotic system 400 to navigate the steerable catheter 450 using, at least in part, motor controls output by the machine-learned model 422. The control processor 410 is configured to control the actuator or motor 444. The machine-learned model 422 is applied by the processor 410. The machine-learned model 422 is configured by previous training to predict a position and/or velocity of the catheter or actuator or motor 444 to implement a user command given an environment of the steerable catheter 450.

In one implementation, the machine-learned model 422 is a recurrent neural network. The machine-learned model 422 predicts in response to input of a sequence of past actuator states and/or past positions of the tip. Other inputs may be provided, such as the user command. The prediction being output is a future sequence of the positions and velocities of the actuator or motor 444 or catheter 450. Other characteristics for motor control may be output.

The machine-learned model 422 outputs the entire or full motor control. In other implementations, the machine-learned model 422 outputs an adjustment or refinement. For example, a kinematics and/or hysteresis model, implemented by the control processor 410, convert calibration information and/or the user command into motor control. The machine-learned model 422 provides a further motor control in the form of further catheter position or adjustment in motor space, which is combined with the other motor control for controlling the motor or actuator 444. The refinement by the machine-learned model 422 accounts for the environment of the catheter 450 within the patient.

The control processor 410 may be configured to use the output of the machine-learned model for additional assistance to the surgeon. The magnitude of the position, velocity, and/or another motor operation parameter and/or the probability for the motor operation parameter output by the machine-learned model 422 are used to generate additional assistance. The additional assistance may be derived environmental information, safety information, and/or boundary information. The output, as a prediction, of the machine-learned model 422, such as magnitude and/or probability, for one or more future times are used to estimate the additional assistance, which is then displayed on the display 430 to the surgeon.

Various illustrative embodiments are summarized below. Any features or aspects in one type of illustrative embodiment (e.g., method or system) may be provided in other types (e.g., computer program product, method, system, and/or non-transitory computer readable medium). Any combination of features or aspects of the illustrative embodiments may be used.

Illustrative embodiment 1. A method for robotic control of a tendon driven continuum mechanism, the method comprising: receiving a user command to operate the tendon driven continuum mechanism; predicting a motor control by an artificial intelligence, the artificial intelligence predicting in response to input of the user command; and operating a motor with the motor control, the operating of the motor operating the tendon driven continuum mechanism.

Illustrative embodiment 2. The illustrative embodiment of claim 1 further comprising operating the motor with the user command wherein the motor control is added in addition to the user command as a fine tuning of the motor operation.

Illustrative embodiment 3. The illustrative embodiment of claim 2 wherein operating the motor with the user command comprises operating the motor with the user command input to a kinematics and hysteresis compensator.

Illustrative embodiment 4. The illustrative embodiment of any of claims 2-3 wherein predicting comprises predicting a change as the motor control to account for friction caused by a current curvature and placement of the tendon driven continuum.

Illustrative embodiment 5. The illustrative embodiment of any of claims 2-4 wherein predicting comprises predicting in response to current draw of the motor and distance from an encoder of the motor input to the artificial intelligence with the user command.

Illustrative embodiment 6. The illustrative embodiment of any of claims 2-5 wherein predicting comprises predicting in response to a temporal sequence of motor position and velocity input to the artificial intelligence with the user command.

Illustrative embodiment 7. The illustrative embodiment of any of claims 2-6 wherein predicting comprises predicting future states by the artificial intelligence in response to past states and a current state, the artificial intelligence comprising a recurrent neural network, the past, current, and future states comprising tip position of the tendon driven continuum mechanism.

Illustrative embodiment 8. The illustrative embodiment of any of claims 2-7 wherein predicting comprises predicting by the artificial intelligence comprising a recurrent neural network outputting a sequence of the motor controls as motor motions minimizing a cost under constraints.

Illustrative embodiment 9. The illustrative embodiment of any of claims 2-8 further comprising outputting environment information for the tendon driven continuum mechanism based on a magnitude of the motor control and/or a probability output by the artificial intelligence for the motor control.

Illustrative embodiment 10. The illustrative embodiment of any of claims 2-9 further comprising outputting safety information for the tendon driven continuum mechanism based on a magnitude of the motor control and/or a probability output by the artificial intelligence for the motor control.

Illustrative embodiment 11. The illustrative embodiment of any of claims 2-10 further comprising predicting a future boundary contact by the tendon driven continuum mechanism based on a magnitude of the motor control and/or a probability output by the artificial intelligence for the motor control.

Illustrative embodiment 12. A control system for a steerable catheter, the control system comprising: a robotic manipulator for operation of the steerable catheter, the robotic manipulator comprising an actuator configured to steer the steerable catheter; and a control processor configured to control the actuator, the control using application of a machine-learned model configured to predict a position and/or velocity of the actuator to implement a user command given an environment of the steerable catheter.

Illustrative embodiment 13. The illustrative embodiment of claim 12 wherein the steerable catheter comprises an intracardiac echocardiography catheter with a tendon connected from a tip to the actuator.

Illustrative embodiment 14. The illustrative embodiment of any of claims 12-13 wherein the steerable catheter has a tip, and wherein the machine-learned model comprises a recurrent neural network configured to predict in response to input of a sequence of past actuator states and/or past positions of the tip, the prediction being output of a future sequence of the positions and velocities of the actuator.

Illustrative embodiment 15. The illustrative embodiment of any of claims 12-14 wherein the control processor is configured to control the actuator with a kinematics and hysteresis model in response to the user command, and wherein the machine-learned model outputs a fine-tuning of the control by the kinematics and hysteresis model to account for the environment.

Illustrative embodiment 16. The illustrative embodiment of any of claims 12-15 wherein the control processor is configured to output environment information about the environment of the steerable catheter based on a magnitude of the position and/or velocity of the actuator and/or a probability output by the machine-learned model for the position and/or velocity.

Illustrative embodiment 17. The illustrative embodiment of any of claims 12-16 wherein the control processor is configured to output safety information of the steerable catheter based on a magnitude of the position and/or velocity of the actuator and/or a probability output by the machine-learned model for the position and/or velocity.

Illustrative embodiment 18. The illustrative embodiment of any of claims 12-17 wherein the control processor is configured to predict a future boundary contact by the steerable catheter based on a magnitude of the position and/or velocity of the actuator and/or a probability output by the machine-learned model for the position and/or velocity.

Illustrative embodiment 19. A method for predictive control of a robotic manipulator for a catheter, the method comprising: predicting a sequence of future states of operation of a motor of the robotic manipulator based on a sequence of past states of the motor operation by a machine-learned model; and controlling the motor of the robotic manipulator based on at least one of the predicted future states.

Illustrative embodiment 20. The illustrative embodiment of claim 19 wherein controlling comprises controlling by a kinematics and/or hysteresis model wherein the at least one of the predicted future states fine-tunes the control by the kinematics and/or hysteresis model, the fine-tuning accounting for friction of a tendon of the catheter due to curvature of the catheter in a patient.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A method for robotic control of a tendon driven continuum mechanism, the method comprising:
receiving a user command to operate the tendon driven continuum mechanism;
predicting a motor control by an artificial intelligence, the artificial intelligence predicting in response to input of the user command; and
operating a motor with the motor control, the operating of the motor operating the tendon driven continuum mechanism.

2. The method of claim 1 further comprising operating the motor with the user command wherein the motor control is added in addition to the user command as a fine tuning of the motor operation.

3. The method of claim 2 wherein operating the motor with the user command comprises operating the motor with the user command input to a kinematics and hysteresis compensator.

4. The method of claim 2 wherein predicting comprises predicting a change as the motor control to account for friction caused by a current curvature and placement of the tendon driven continuum mechanism.

5. The method of claim 1 wherein predicting comprises predicting in response to current draw of the motor and distance from an encoder of the motor input to the artificial intelligence with the user command.

6. The method of claim 1 wherein predicting comprises predicting in response to a temporal sequence of motor position and velocity input to the artificial intelligence with the user command.

7. The method of claim 1 wherein predicting comprises predicting future states by the artificial intelligence in response to past states and a current state, the artificial intelligence comprising a recurrent neural network, the past, current, and future states comprising tip position of the tendon driven continuum mechanism.

8. The method of claim 1 wherein predicting comprises predicting by the artificial intelligence comprising a recurrent neural network outputting a sequence of the motor controls as motor motions minimizing a cost under constraints.

9. The method of claim 1 further comprising outputting environment information for the tendon driven continuum mechanism based on a magnitude of the motor control and/or a probability output by the artificial intelligence for the motor control.

10. The method of claim 1 further comprising outputting safety information for the tendon driven continuum mechanism based on a magnitude of the motor control and/or a probability output by the artificial intelligence for the motor control.

11. The method of claim 1 further comprising predicting a future boundary contact by the tendon driven continuum mechanism based on a magnitude of the motor control and/or a probability output by the artificial intelligence for the motor control.

12. A control system for a steerable catheter, the control system comprising:
a robotic manipulator for operation of the steerable catheter, the robotic manipulator comprising an actuator configured to steer the steerable catheter; and
a control processor configured to control the actuator, the control using application of a machine-learned model configured to predict a position and/or velocity of the actuator to implement a user command given an environment of the steerable catheter.

13. The control system of claim 12 wherein the steerable catheter comprises an intracardiac echocardiography catheter with a tendon connected from a tip to the actuator.

14. The control system of claim 12 wherein the steerable catheter has a tip, and wherein the machine-learned model comprises a recurrent neural network configured to predict in response to input of a sequence of past actuator states and/or past positions of the tip, the prediction being output of a future sequence of the positions and velocities of the actuator.

15. The control system of claim 12 wherein the control processor is configured to control the actuator with a kinematics and hysteresis model in response to the user command, and wherein the machine-learned model outputs a fine-tuning of the control by the kinematics and hysteresis model to account for the environment.

16. The control system of claim 12 wherein the control processor is configured to output environment information about the environment of the steerable catheter based on a magnitude of the position and/or velocity of the actuator and/or a probability output by the machine-learned model for the position and/or velocity.

17. The control system of claim 12 wherein the control processor is configured to output safety information of the steerable catheter based on a magnitude of the position and/or velocity of the actuator and/or a probability output by the machine-learned model for the position and/or velocity.

18. The control system of claim 12 wherein the control processor is configured to predict a future boundary contact by the steerable catheter based on a magnitude of the position and/or velocity of the actuator and/or a probability output by the machine-learned model for the position and/or velocity.

19. A method for predictive control of a robotic manipulator for a catheter, the method comprising:
predicting a sequence of future states of operation of a motor of the robotic manipulator based on a sequence of past states of the motor operation by a machine-learned model; and
controlling the motor of the robotic manipulator based on at least one of the predicted future states.

20. The method of claim 19 wherein controlling comprises controlling by a kinematics and/or hysteresis model wherein the at least one of the predicted future states fine-tunes the control by the kinematics and/or hysteresis model, the fine-tuning accounting for friction of a tendon of the catheter due to curvature of the catheter in a patient.
